(19) United States
(12) Patent Application Publication
(10) Pub. No.: US 2010/0215787 A1
(43) Pub. Date: Aug. 26, 2010

(54) Composition For The Treatment Of Gastrointestinal Diseases

(75) Inventors: Hayley Bennett, Stockport (GB); Ian Stanley Roberts, Cheshire (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: THE UNIVERSITY OF MANCHESTER, Manchester (GB)

(21) Appl. No.: 12/513,208

(22) PCT Filed: Nov. 2, 2007
(86) PCT No.: PCT/GB07/04171

§ 371 Date: Dec. 7, 2009

(30) Foreign Application Priority Data
Nov. 3, 2006 (GB) 0622137-8

(51) Int. Cl. A61K 36/81; A61P 31/04; A61P 1/00; A61P 29/00

(52) U.S. Cl. 424/773

(57) ABSTRACT

The present invention relates to compositions (including medicaments and nutritional products) for use in the prevention or treatment of gastrointestinal disease. Such compositions comprise a therapeutically effective amount of an alcohol soluble fraction derivable from potato juice.

The present invention relates to the prevention and treatment of gastrointestinal diseases and in particular, to the prevention and treatment of gastrointestinal diseases with extracts derived from edible plants.

Gastritis covers a variety of different conditions that all cause inflammation of the stomach. Inflammation may result from an infection with bacteria but other factors, including traumatic injury and regular use of certain pain relievers, can also contribute to gastritis.

In spite of the many conditions associated with gastritis, the signs and symptoms of the disease are very similar: a burning pain in the upper abdomen and occasionally, bloating, belching, nausea or vomiting. In some cases, gastritis can lead to ulcers and an increased risk of stomach cancer.

A peptic ulcer is a sore on the lining of the stomach or duodenum. One cause of peptic ulcer is bacterial infection, but some ulcers are caused by long-term use of nonsteroidal anti-inflammatory agents (NSAIDs), like aspirin and ibuprofen. In a few cases, cancerous tumors in the stomach or pancreas can cause ulcers.

Helicobacter pylori (H. pylori) is a type of bacteria that is a major cause of chronic gastritis and plays a pivotal role in the development of both gastric and duodenal ulcers (1). H. pylori weakens the protective mucous coating of the stomach and duodenum, which allows acid to get through to the sensitive lining beneath. Both the acid and the bacteria irritate the lining and cause a sore, or ulcer.

H. pylori is able to survive in stomach acid because it secretes enzymes that neutralize the acid. This mechanism allows H. pylori to make its way to the “safe” area; the protective mucous lining. Once there, the spiral shape of the bacterium helps it burrow through the lining. Chronic infection with this organism is associated with increased risk for the development of gastric adenocarcinoma and gastric lymphoma (2). Adhesion of H. pylori to the gastric epithelium has been shown to influence disease severity (3). It promotes translocation of a cytotoxin-associated antigen A (CagA) into the host cell, via the cag pathogenicity island encoded type IV secretion system (TFSS), causing subversion of host cell functions and increasing the risk of gastric cancer development (4). As such, the ability to adhere to host gastric mucosa is a prerequisite for both initial colonisation and continued survival and proliferation with the subsequent development of host pathology.

Treatment of H. pylori infections usually follows a seven or ten day triple therapy regime, including a proton pump inhibitor such as omeprazole or rabeprazole, together with commonly used antibiotics such as clarithromycin, metronidazole and amoxicillin (5). Although effective, such a regime can be undesirable from both a cost perspective and also as adverse side effects such as diarrhoea metallic taste and allergic reaction are known to occur. Resistance to clarithromycin and metronidazole is a major cause of treatment failure with 10 to 20% of patients failing to successfully eradicate the organism following therapy (6). This high failure rate has lead to a number of non-antibiotic therapies being considered as potential alternatives for treatment of H. pylori infection (7).

Several reports have described the activity of plant extracts and their isolated constituents against H. pylori. For example the use of vegetables such as broccoli for the treatment of gastritis is well known (8). In addition the active constituent of broccoli, the isothiocyanate sulforaphane, showed activity against H. pylori including antibiotic resistant strains (9). Furthermore the inhibition of growth and urease production of H. pylori by alcoholic extracts of the shallot Allium ascalonicum (10) and by oregano and cranberry (11) has also been shown. Other plants that have been shown to exhibit toxicity towards H. pylori are the North American herbal medicines Sanguinaria canadensis and Hydrastis Canadensis (12), the Asian herb Plumbago zeylanica (13), a range of Greek traditional medicines (14), and green tea extract (15). More recently a report described the antibacterial activity of nutmeg, parsley, tarragon, long pepper, sage and cinnamon against H. pylori (16). However, many of these culinary and medicinal herbs exhibit a broad spectrum of activity with toxicity having been shown against both Gram-positive and Gram-negative bacteria (17). Thus the consumption of such herbs and their products may also have adverse effects on the probiotic micro flora, many of which are known to be beneficial (18). Therefore, in order to be efficacious natural anti-H. pylori agents should exhibit a narrow spectrum of activity and ideally be selective towards H. pylori alone.

It is an object of the present invention to provide alternative non-antibiotic compositions useful in the prevention or treatment of gastrointestinal diseases, in particular gastric and duodenal ulcers.

According to a first aspect of the present invention there is provided a method of making a composition for use in the prevention or treatment of gastrointestinal diseases comprising the steps of adding an alcohol to potato juice to form an alcohol soluble fraction and an alcohol insoluble precipitate and recovering the alcohol soluble fraction.

The composition prepared by said method is an alcohol soluble fraction that may be used in the prevention or treatment of gastrointestinal diseases. Preferably, ethanol is used for the precipitation. Preferably, the fraction comprises a greater than or equal to 3 kDa soluble fraction, most preferably an equal to or greater than 500 Da to less than or equal to 1 kDa soluble fraction derivable from potato juice in a therapeutically effective amount.

According to a second aspect of the present invention there is provided the use of an alcohol soluble fraction derivable from potato juice for the manufacture of a medicament for the prevention or treatment of gastrointestinal diseases.

According to a third aspect of the present invention there is provided a method for the treatment of gastrointestinal diseases comprising administering to a subject in need of such treatment a therapeutically effective amount of an alcohol soluble fraction derivable from potato juice.

According to a fourth aspect of the present invention there is provided a fraction comprising alcohol and a soluble fraction derivable from potato juice wherein the fraction derivable from potato juice comprises a greater than or equal to 3 kDa soluble fraction, most preferably an equal to or greater than 500 Da to less than or equal to 1 kDa soluble fraction.

More preferably, the alcohol plus potato juice fraction is the range 8 kDa to 14 kDa, especially 10 kDa to 12 kDa. Preferably, the fraction is positively charged at pH 8. It is preferable for the fraction to include at least part of a protein, or a polypeptide. It is preferable for the fraction to contain one or more of the following: reducing sugars, protein and components with primary amino groups.

It is to be appreciated that the soluble extract according to the present invention may be in pure form or alternatively it may also be mixed with other compounds, provided those compounds do not inhibit the anti-bacterial properties of the extract according to the invention.

The alcohol, preferably ethanol, soluble fraction has been found to have bactericidal activity against H. pylori and thus have efficacy for preventing or treating gastritis and duodenal ulcers. Furthermore, advantageously the extract had no detectable effect on the viability of a range of other bacterial species thus indicating that treatments according to the present invention should have no adverse effect on the rest of the normal host gut flora and the problems associated with the development and spread of drug resistance.

Thus, a fifth aspect of the present invention provides an antibacterial agent comprising an alcohol soluble fraction derivable from potato juice. The antibacterial agent is particularly effective against H. pylori.

The alcohol soluble extract according to the invention may be prepared in its simplest form by homogenising potato juice, decanting off the aqueous supernatant and centrifuging the supernatant. This extract may, be concentrated, enriched or condensed by, for example, standard techniques, e.g. evaporation under reduced pressure. Examples of concentrates are those which are at least 2-fold concentrated, more usually, at least 4-fold, for example at least 8-fold, or at least 20-fold, or at least 100-fold, or at least 200-fold, or at least 1000-fold.

The concentrated potato juice is preferably subjected to more than 1 precipitation with alcohol, more preferably at least four precipitations, the alcohol preferably being ethanol. Preferably, at least 70% ethanol is used, especially 96% ethanol. Preferably, 1 volume of concentrated potato juice is subjected to precipitations with 4 volumes of ethanol.

Any suitable method may be used to separate the aqueous active solution from the insoluble precipitate, including, for example, centrifugation or molecular weight filtration.

The crude potato juice according to the present invention may be prepared from potato juice by one or more of the following steps:

(a) maceration of potato tuber, optionally freshly peeled; and

(b) centrifugal filtration of the resulting aqueous juices.

Appropriate organic solvents may be used to aid purification of the alcohol soluble fraction, as may chromatographic procedures, such as ion exchange rains and further size exclusion, for example of Superdex™.

Preferably, the alcohol soluble fraction is further purified by means of HPLC separation, in particular being subjected to anion HPLC at pH 8 to bind negatively charged components, collecting the non-bound components and treating said non-bound components with cation HPLC at pH 8 to bind positively charged components, eluting the positively charged components from the column and collecting the eluted fraction as the purified extract.

In a preferred embodiment of the present invention there is provided a method of making an anti-bacterial agent comprising the steps of
preparing an aqueous extract of potato juice;
adding an alcohol to the aqueous extract to form an alcohol soluble fraction and an alcohol soluble precipitate;
recovering the alcohol soluble fraction;
treating said fraction with an anion exchange resin to bind negatively charged components and collecting the non-bound components from the resin;
treating said non-bound components with a cation exchange resin to associate positively-charged components with said resin;
eluting said positively-charged components from said resin with an eluting solution to obtain an eluate having anti-bacterial properties;
collecting and, optionally concentrating, said eluate.

The alcohol soluble fraction may optionally be freeze or spray dried to make a powdered fraction according to the invention. A crude preparation may be used substantially “neat” or even diluted. When this is the case the supernatant (whether diluted or not) may be mixed with a number of other agents that may be added for nutritional reasons, medical reasons or even for the purposes of adjusting the palatability of the extract for consumption by the subject being treated.

For instance, the extract may be formulated with a food or drink product to provide a functional food having the characteristic that it contains an alcohol soluble fraction of potato juice that prevents or relieves the symptoms of gastritis. For example, the extract may be formulated as a diary product using milk as the raw material (for example, milk, a milk shake, yoghurt, yoghurt drink or dairy supplement) to produce a palatable drink/beverage with the added benefit that it contains the active fraction and therefore will be highly suitable as a refreshment for sufferers of gastritis. It is to be appreciated that the extract could be formulated as an alternative beverage, such as a fruit juice, albeit this is unlikely to be the desired choice of a person suffering from gastritis.

Alternatively, the crude preparation may be included in a nutritional liquid for enteral feeding. For instance, the alcohol soluble fraction may be mixed with saline or an aqueous solution (other vitamins, minerals and nutrients may be included) for enteral feeding of subjects.

It will be appreciated that concentration of the crude preparation may be required or alternatively a powder composition may be desired. When this is the case the crude extract will need to be concentrated/dehydrated.

The alcohol soluble fraction may be formulated as powders, granules or semisolids for incorporation into capsules. For presentation in the form of a semisolid, the extract can be dissolved or suspended in a viscous liquid or semisolid vehicle such as a polyethylene glycol, or a liquid carrier such as a glycol, for example, propylene glycol, or glycerol or a vegetable or fish oil, for example an oil selected from olive oil, sunflower oil, safflower oil, evening primrose oil, soya oil, cold liver oil, herring oil, etc. This may then be filled into capsules of either the hard gelatine or soft gelatine type or made from hard or soft gelatine equivalents, soft gelatine or gelating-equivalent capsules being preferred for viscous liquid or semisolid fillings.

Powders comprising the alcohol soluble fraction according to the invention are particularly useful for making pharmaceutical or nutritional products that may be used to prevent or treat gastrointestinal diseases, in particular gastric and duodenal ulcers.

Freeze-drying or spray drying represent preferred methods for producing a powder comprising the extract according to the invention. Spray drying results in free-flowing granular powder mixes with good flow properties and quick dissolving characteristics.

Powdered extract may be reconstituted as a clear/translucent low viscosity drink/beverage. Reconstitution may be into water or dairy or fruit juices as discussed above. It will be appreciated that the powder may be packaged in a sachet and reconstituted as a drink by a subject when required or desired.

Powder mixes represent preferred embodiments of the invention. Such mixes comprise powdered extract (as described above) mixed with further ingredients. Such ingredients may be added for nutritional or medical reasons or for improved palatability. The powdered extract may be mixed with granulated sugars of varying particle sizes to obtain free-flowing powder mixes of varying sweetness.

Alternatively natural sweeteners or artificial sweeteners (for example, aspartame, saccharin and the like) may be mixed with the powdered extract for reconstitution as a low calorie/reduced calorie sweetened drink. The powder mix may comprise a mineral supplement. The mineral may be any one of calcium, magnesium, potassium, zinc, sodium, iron, and their various combinations.

Powder mixes may also contain buffering agents such as citrate and phosphate buffers, and effervescent agents formed from carbonates, for example, bicarbonates such as sodium or ammonium bicarbonate, and a solid acid, for example citric acid or an acid citrate salt.

The alcohol soluble fraction can be presented as food supplements or food additives, or can be incorporated into foods, for example functional foods or nutriceuticals. Such products may be used as staple foods as well as under circumstances where there may be a clinical need.

The powders may be incorporated into bakery or cereal products, such as snack food bars for example fruit bars, nut bars and cereal bars. For presentation in the form of snack food bars, the powder can be admixed with any one or more ingredients selected from dried fruits such as sundried tomatoes, raisins and sultanas, ground nuts or cereals such as oats and wheat.

It will be appreciated that the alcohol soluble fraction may advantageously be formulated as a pharmaceutical for use as a medicament (requiring a prescription or otherwise).

The powdered fraction or concentrated liquid fraction may also be incorporated into tablets, lozenges, sweets or other food-stuffs for oral ingestion. It will also be appreciated that such powdered fraction or concentrated liquid fraction may be incorporated into slow-release capsules or devices which may be ingested and are able to release the fraction into the intestines over a long period of time.

The fraction may also be microencapsulated. For instance encapsulation may be by calcium-alginate gel capsule formation. Kappa-carrageenan, gellan gum, gelatin and starch may be used as excipients for micro-encapsulation.

Crude preparations, liquid concentrates, powders and the like may be combined with known therapeutic agents for treating gastritis. As such the extract according to the invention may be used in a very effective combination therapy. It will be appreciated that the extract fibre in solution may act as an ideal vehicle for other therapeutic agents for treating gastritis.

The alcohol soluble fraction may also be included in combination/synbiotic therapies that include a probiotic portion. The bacteria contained within many probiotic mixtures do not possess adhesive properties so would not be affected by the inclusion of the active fraction.

The compositions of the invention can be presented in the form of unit dosage forms containing a defined concentration of the extract. Such unit dosage forms can be selected so as to achieve a desired level of biological activity.

The amount of extract required by a subject is determined by biological activity and bioavailability that in turn depends on the formulation, mode of administration, the physicochemical properties of the extract and whether it is being used as a monotherapy or in a combined therapy. Generally, a daily dose for a human adult should be between 0.1 g and 100 g of freeze-dried or spray-dried powder (however formulated), more preferably the daily dose is between 1 g and 30 g (for example, about 5 g, 10 g, or 15 g as required).

The extract is particularly useful when included in pharmaceutical formulation such as a tablet or a capsule. Such formulations may be required to be enterally-coated if bioavailabilty dictates this. Known procedures, such as those conventionally employed by the pharmaceutical industry (for example, in vivo experimentation, clinical trials etc), may be used to establish specific formulations of pharmaceutical compositions and precise therapeutic regimes (such as daily doses of the compounds and the frequency of administration).

It will be appreciated that conventional “nutriceutical” procedures may be employed to create liquid drinks, powder mixes and food-stuffs comprising the alcohol soluble fraction.

Daily doses may be given as a single administration (for example, a daily tablet for oral consumption or as a single liquid drink). Alternatively the extract used may require administration twice or more times during a day. As an example, a 100 ml orange drink containing between 0.1 and 20 g of spray dried extract, preferably between 0.3 and 10 g of spray dried extract and more preferably between 0.5 and 3.0 g may be used to quench thirst at regular intervals throughout the day and thereby deliver a recommended dose.

It will be appreciated that nutritional products supplemented with the alcohol soluble potato extract represent an ideal means for providing subjects with, or at risk of developing, gastritis or other gastrointestinal disease with the extract according to the present invention. Therefore, according to a fifth aspect of the present invention there is provided a nutritional product for use in the prevention or treatment of gastrointestinal diseases comprising a therapeutically effective amount of an alcohol soluble fraction derivable from potato juice.

The nutritional product may comprise:
(a) a clear, low viscosity, water-like, stable, ready-to-use, bottled, carbonated or non-carbonated drink; or a concentrated clear liquid for reconstitution containing an alcohol soluble fraction derivable from potato juice;
(b) a powder/granular mix to be reconstituted with water or any other orally ingestible liquid as a drinkable liquid, containing an alcohol soluble fraction derivable from potato juice; or
(c) a powder/granular mix mixed into a food stuff (for example, a cereal or chocolate bar, lozenge or the like).

The nutritional product may be as described above and may or may not contain water soluble vitamins, additional mineral supplements, nutritional compounds, antioxidants or flavourings.

The present invention will be further illustrated, by way of the following examples and with reference to the accompanying drawings in which:—

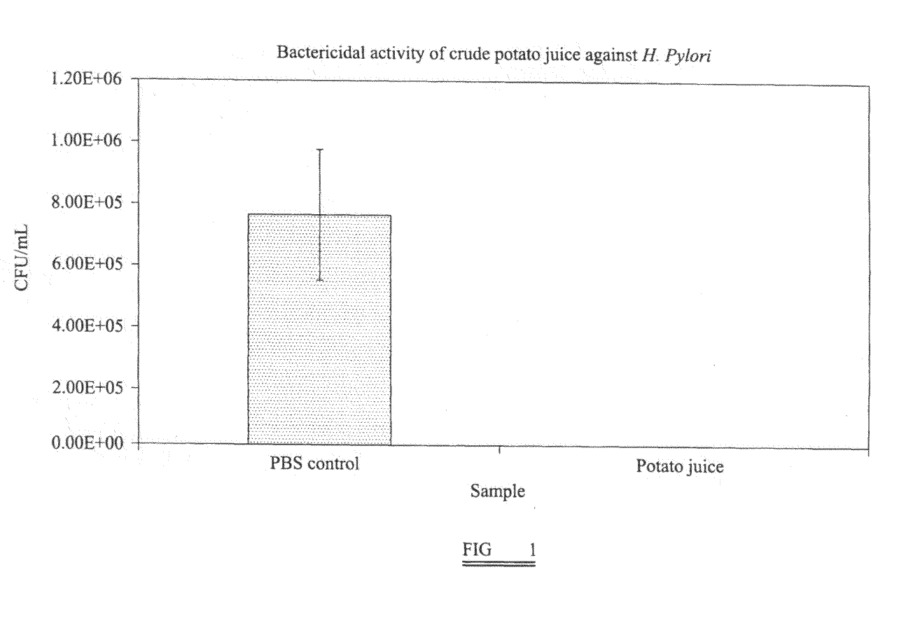

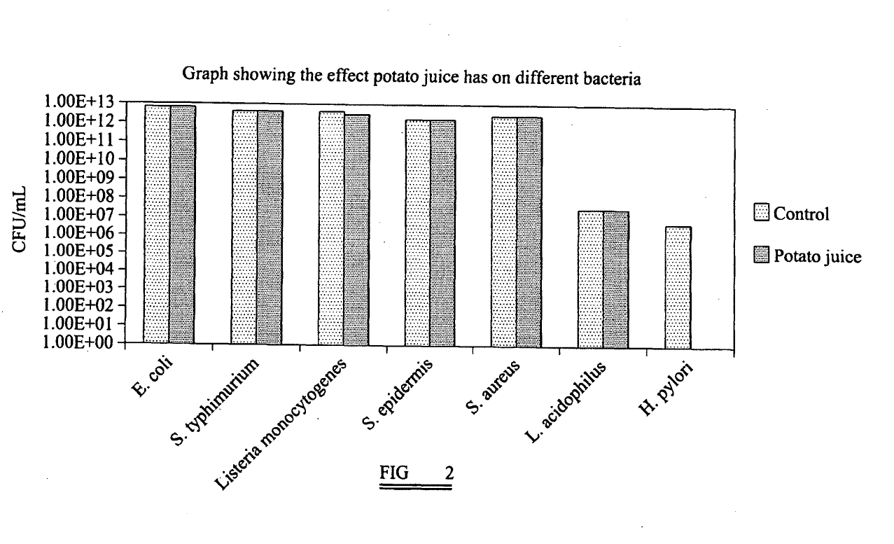

FIG. 1 is a graph illustrating the bactericidal activity of potato juice;

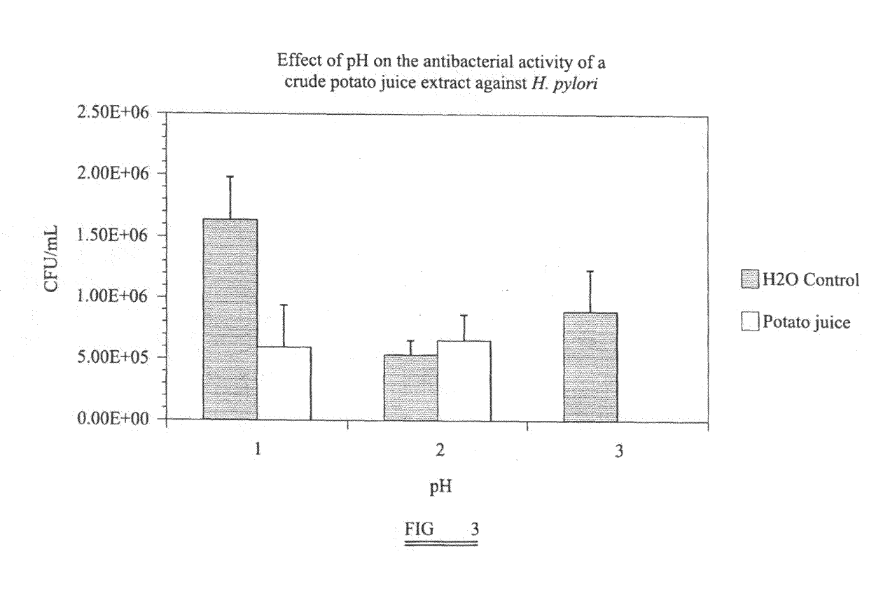

FIG. 2 is a graph showing the effect potato juice has on different bacteria;

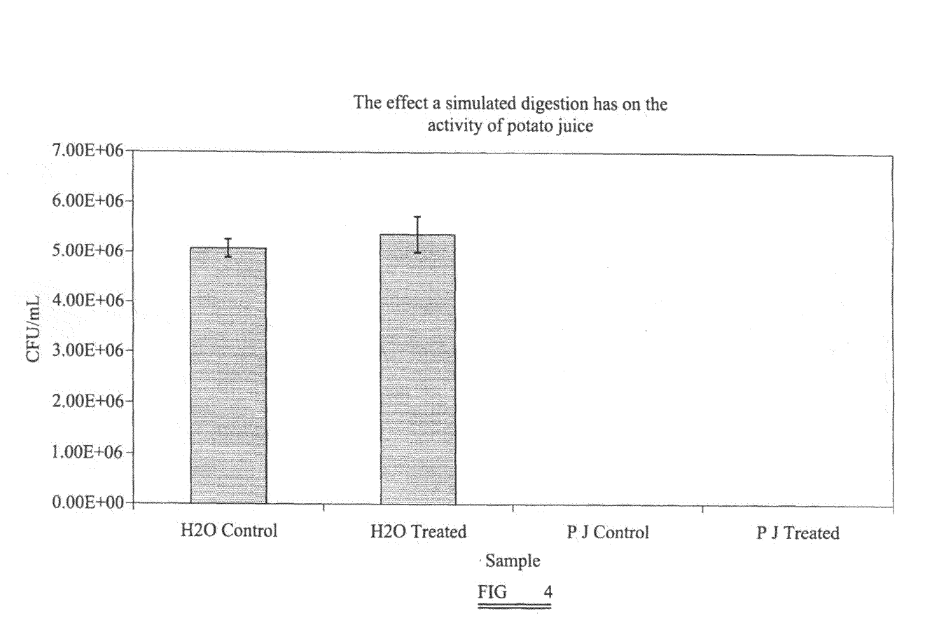

FIG. 3 is a graph illustrating the effect of pH on the anti-bacterial activity of a crude potato juice extract against H. pylori;

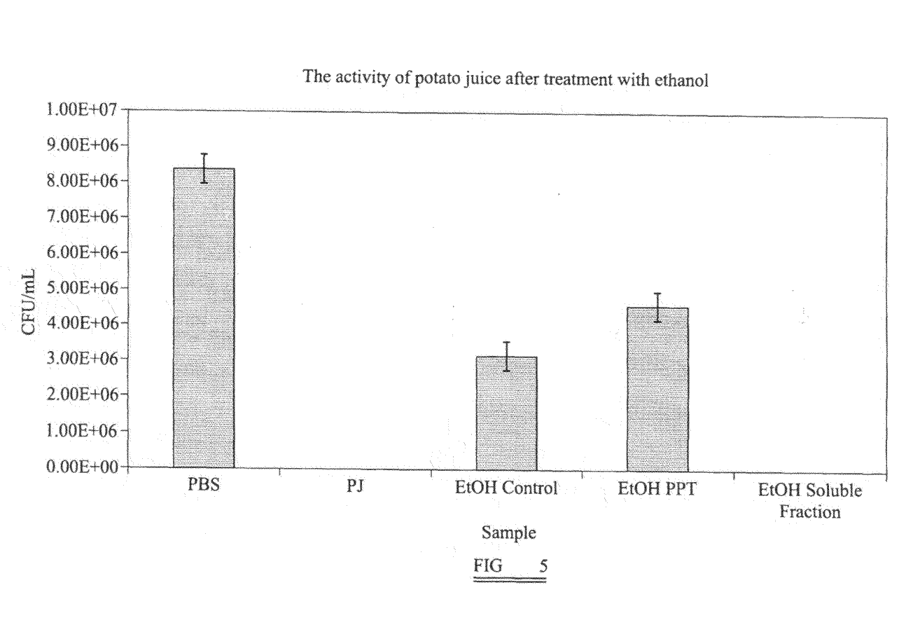

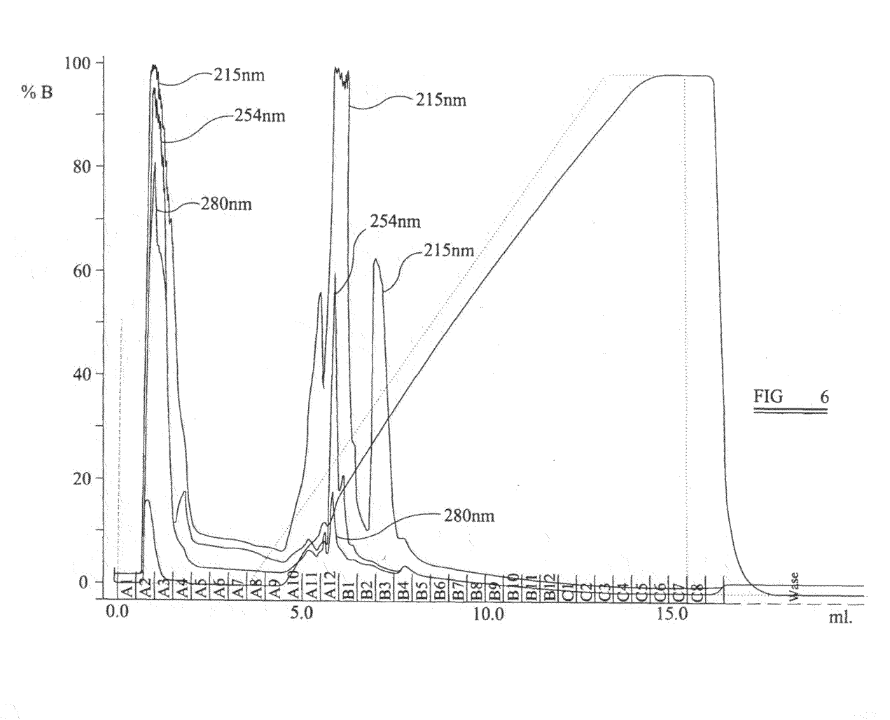

FIG. 4 is a graph illustrating the effect of a simulated digestion on the activity of potato juice;

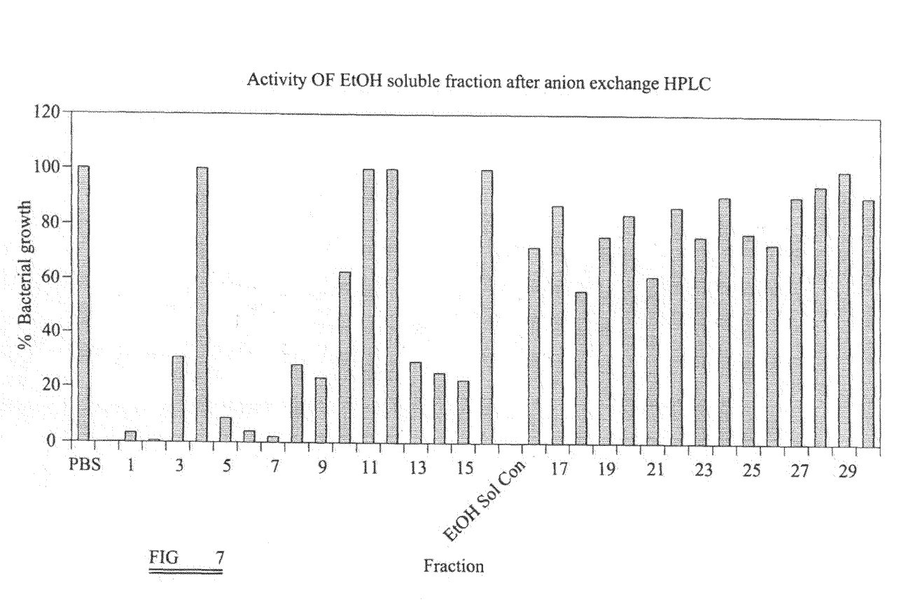

FIG. 5 is a graph illustrating the activity of potato juice (PJ) after treatment with ethanol;

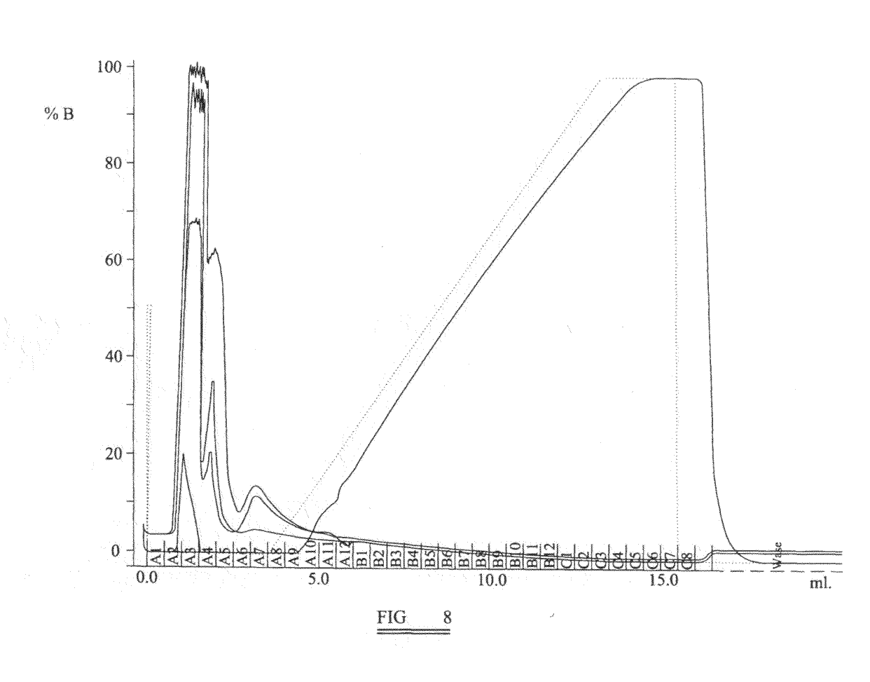

FIG. 6 is an anion exchange HPLC chromatogram of the EtOH soluble fraction;

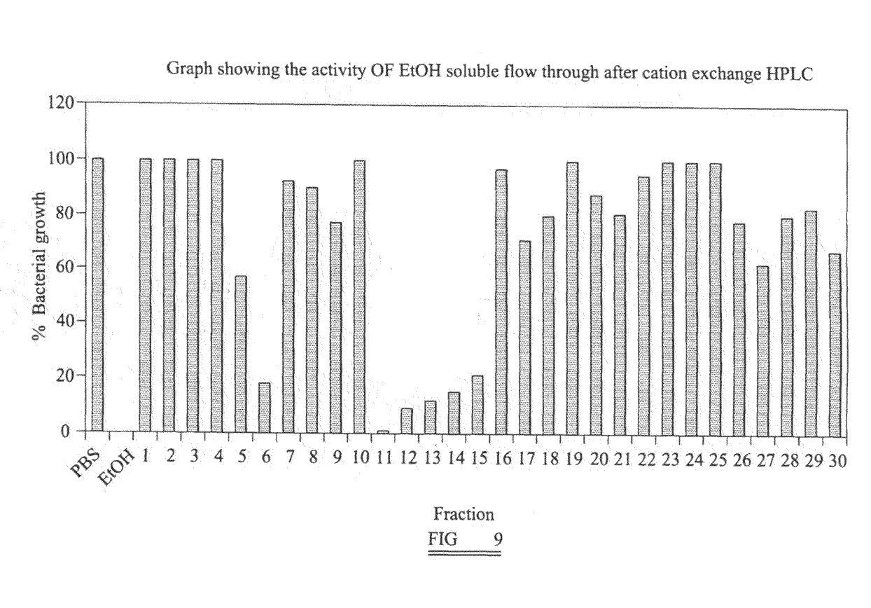

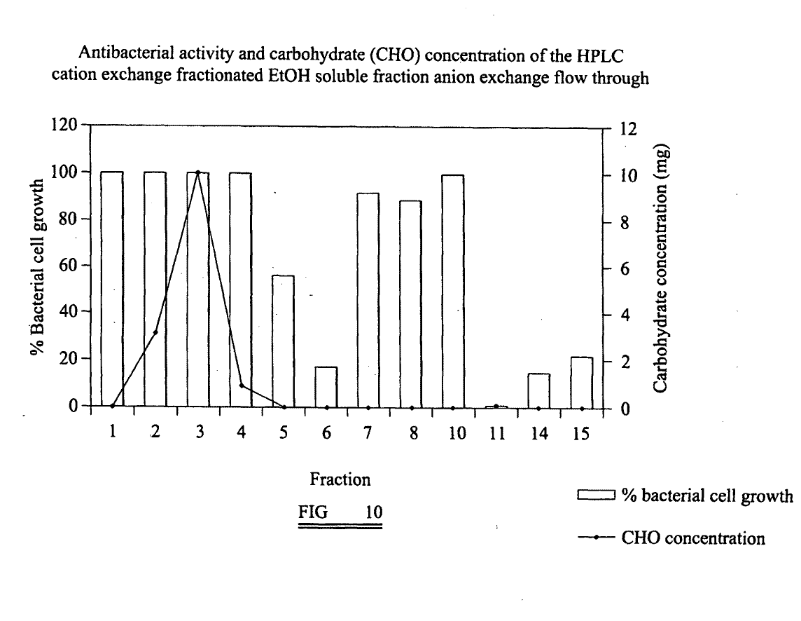

FIG. 7 is a graph showing the activity profile of the EtOH soluble fraction after anion exchange HPLC;

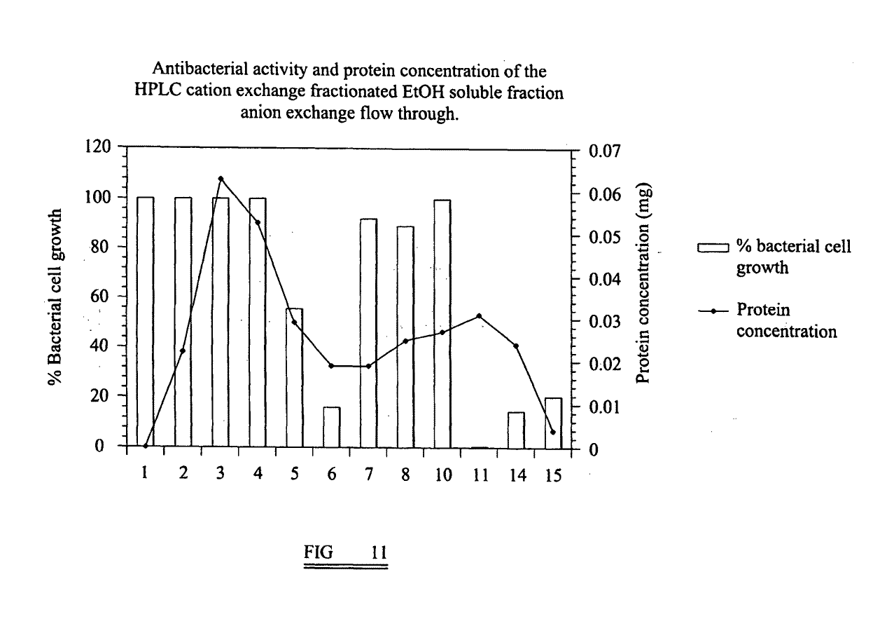

FIG. 8 is a cation exchange HPLC chromatogram of bulk isolated EtOH soluble fraction anion exchange flow through;

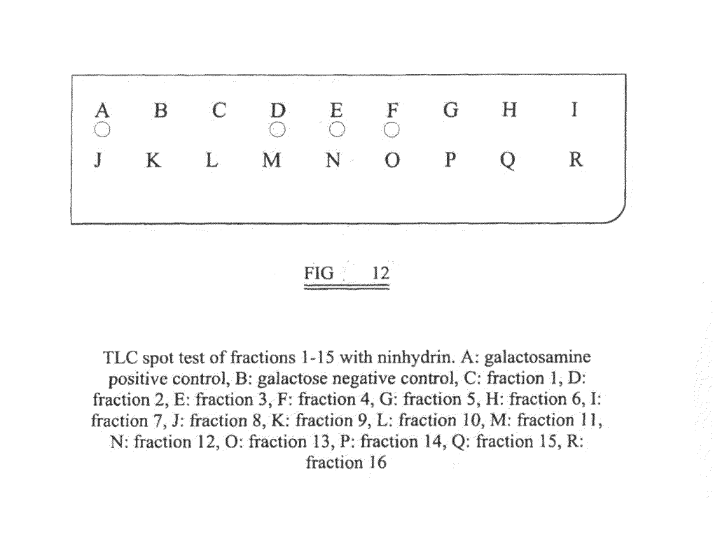

FIG. 9 is a graph showing the activity profile of cation exchange separation of the anion exchange flow through of the EtOH soluble fraction;

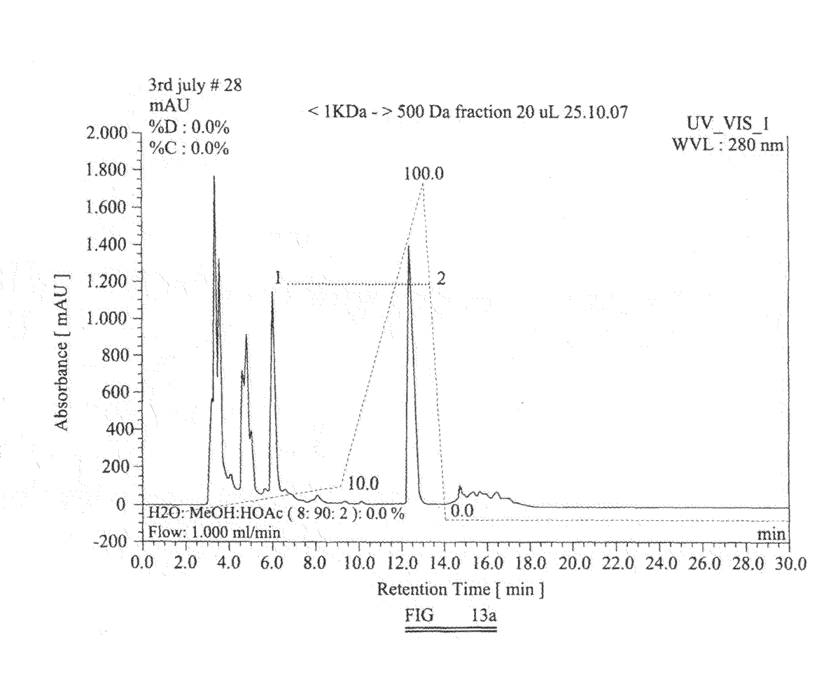

FIG. 10 is a graph illustrating anti bacterial activity and carbohydrate concentration of the HPLC cation exchange fractionated EtOH soluble fraction anion exchange flow through;

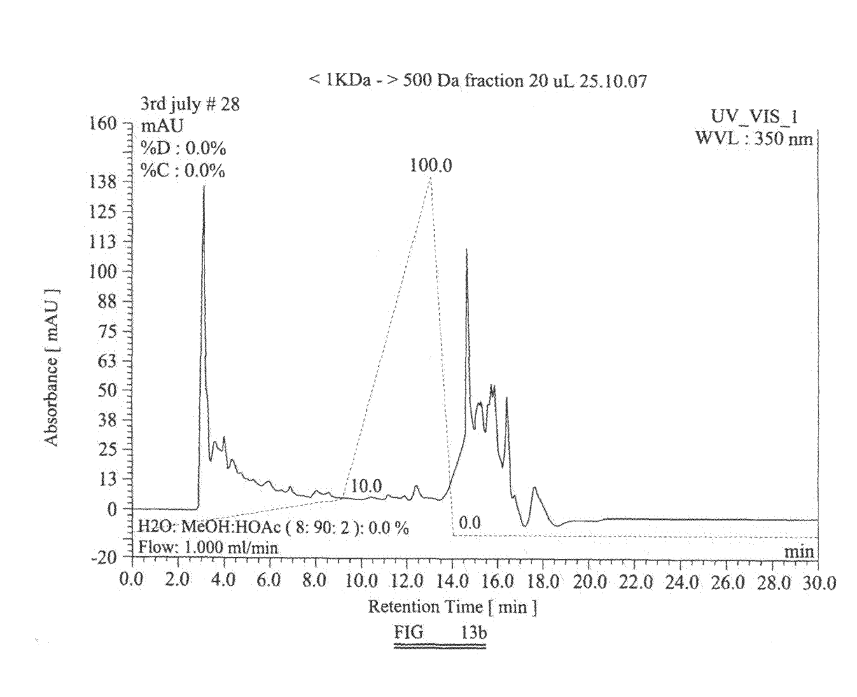

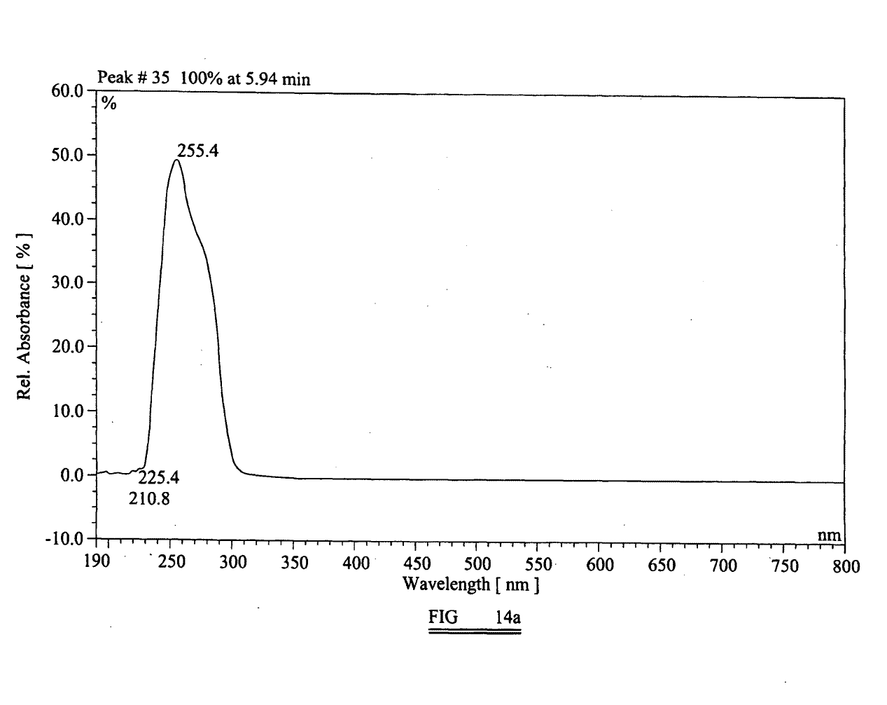

FIG. 11 is a graph illustrating antibacterial activity and protein concentration of the HPLC cation exchange fractionated EtOH soluble fraction anion exchange flow through;

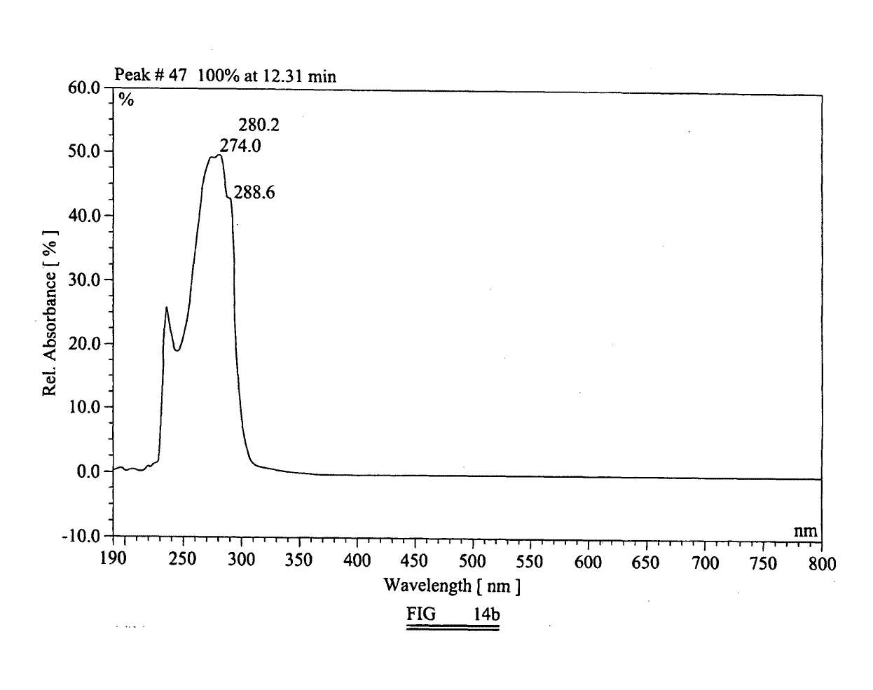

FIG. 12 is a TLC spot test of fractions 1 to 15 of the cation exchange fractionated EtOH soluble fraction anion exchange flow through;

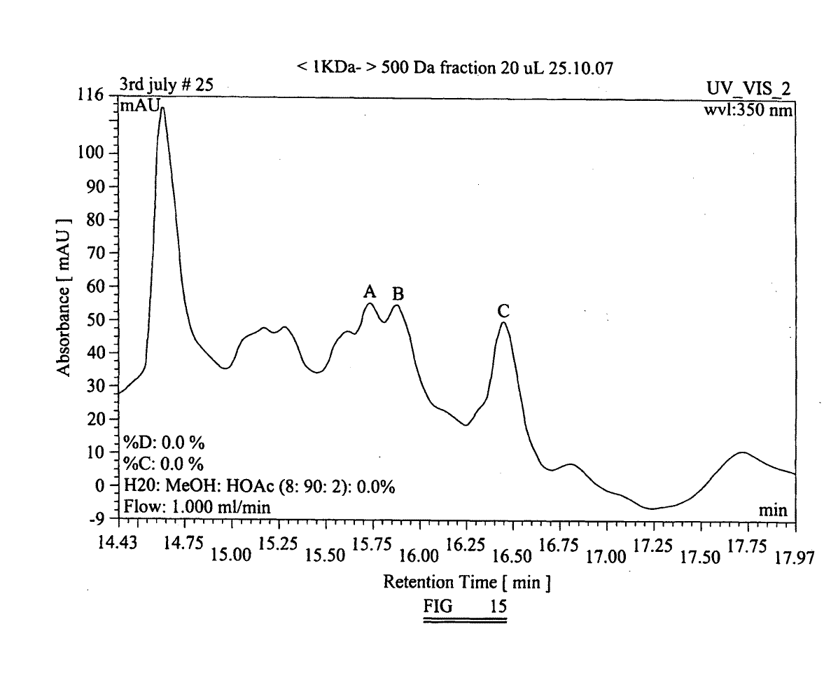

FIG. 13a is a HPLC chromatogram of the less than or equal to 1 kDa to greater than or equal to 500 Da fraction on a Gemini C₁₈ reversed phase column. UV detection at 280 nm 1, 2: peaks 1 and 2;

FIG. 13b is a HPLC chromatogram of the less than or equal to 1 kDa to greater than or equal to 500 Da fraction on a Gemini C₁₈ reversed phase column. UV detection at 350 nm;

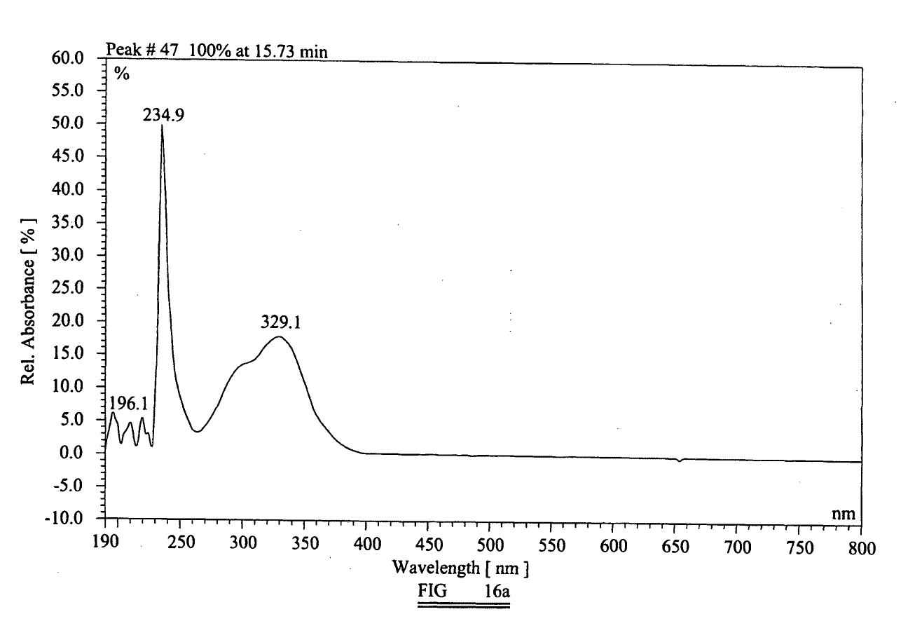

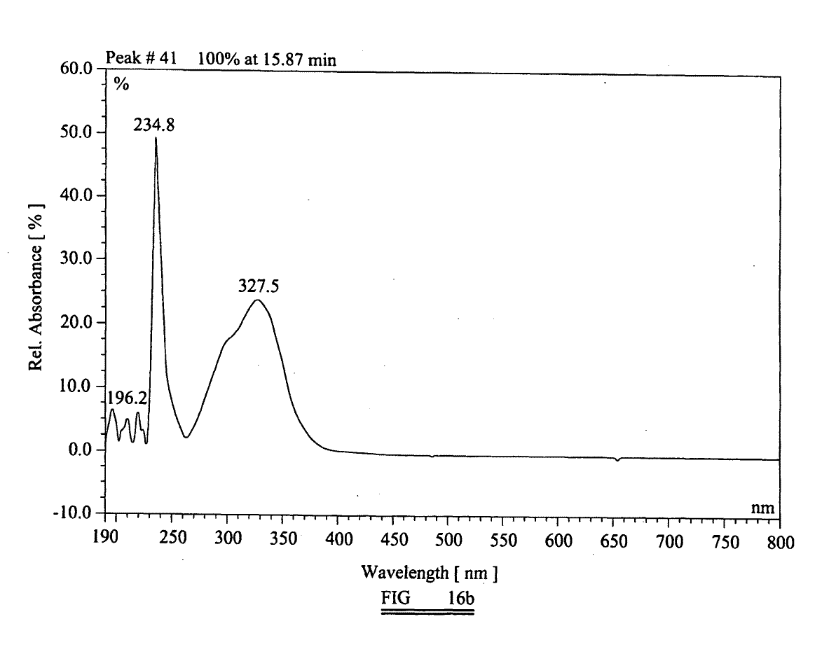

FIG. 14a is a UV spectrum of peak 1 at 280 nm;

FIG. 14b is a UV spectrum of peak 2 at 280 nm;

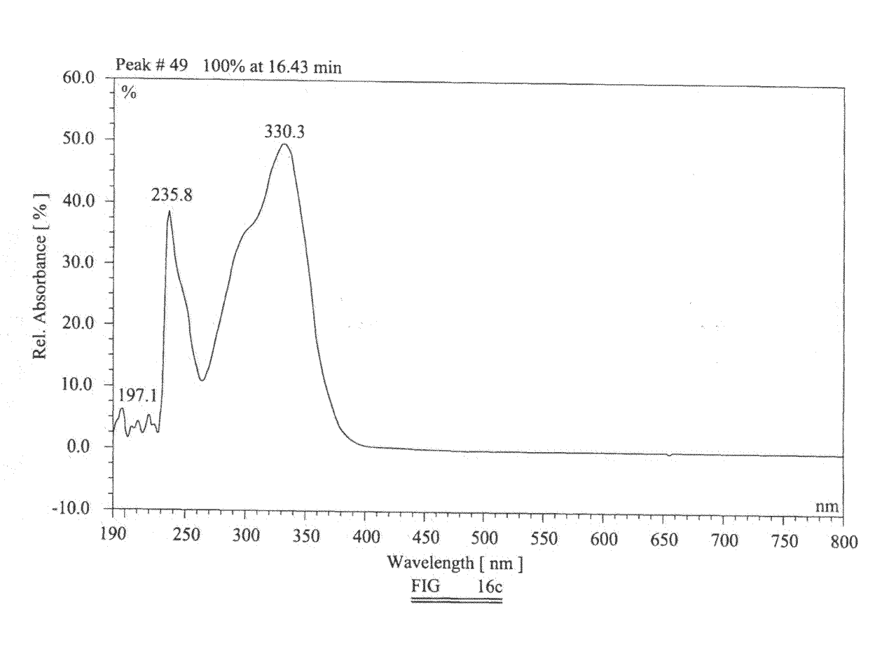

FIG. 15 is a close up view of the components eluting between 14 to 17 minutes with detection at 350 nm. A-C: peaks A-C;

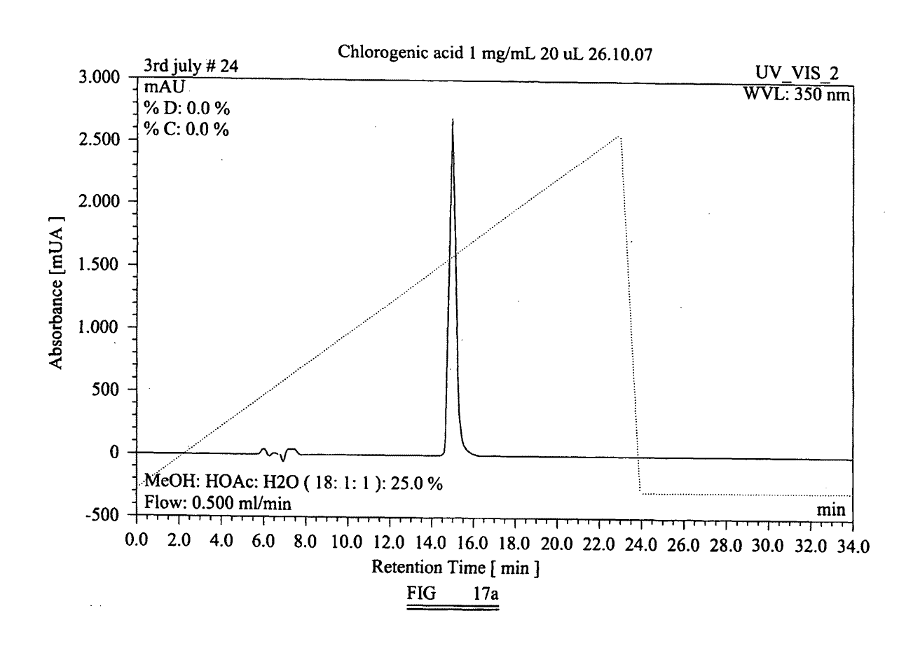

FIG. 16a is a UV spectrum of peak A at 350 nm;

FIG. 16b is a UV spectrum of peak B at 350 nm;

FIG. 16c is a UV spectrum of peak C at 250 nm;

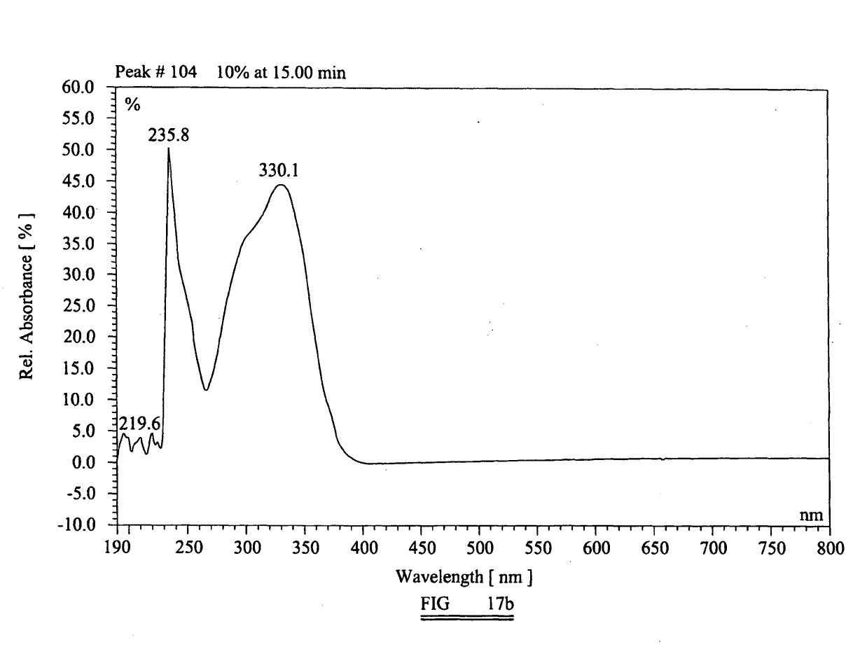

FIG. 17a is a HPLC chromatogram of chlorogenic acid marker at 350 nm; and

FIG. 17b is a UV spectrum of chlorogenic acid marker at 350 nm.

The inventors conducted studies to determine if potato juice contains bacterial activity towards Helicobacter pylori and a range of other bacterial species.

### Preparation of Extract:

Crude potato juice: Peeled and washed potatoes were weighed, ground in a blender, sieve filtered and centrifuged at 8,000 to 13,000 rpm for 5 to 20 minutes. The soluble extract was then filtered through a 0.22 μm membrane and the extract was stored at 80° C. until required for further analysis.

### EXAMPLE 1

### Potato Juice was Assayed Against a Range of Different Bacteria to Assess Whether it Had a Narrow or Broad Spectrum of Activity

The following bacteria were used: Helicobacter pylori, Escherichia coli, Lactobacillus acidophilus, Staphylococcus aureus, staphylococcus epidermidis, Listeria monocytogenes and Salmonella enterica sv. Typhiiuurium. H, pylori was cultured under microaerophilic conditions at 37° C. on Columbia agar (OXOID CM331) containing 5% v/v defibrinated horse blood (TCS Biosciences HB035) and 0.25% v/v H. Pylori selective supplement (OXOID SR0147E). Cultures were passaged onto fresh media every 4 days. L. acidophilus stock cultures were prepared in 50% glycerol from cultures initiated under microaerophilic conditions in MRS broth (OXOID CM0359) and assays were done using MRS broth and MRS Agar (OXOID CM0361). All other bacteria were incubated aerobically at 37° C. on Luria-Bertani broth/agar or on Mueller-Hinton broth/agar (OXOID CM405) and were initiated from laboratory glycerol stock cultures.

### Agar Well Diffusion Assay:

H. pylori cells were resuspended in PBS (pH 8) to an OD_{600 nm}=0.25 (10⁷ cfu/mL). All other bacteria were adjusted to an OD_{625 mm}=0.1 (10⁷-10⁸ cfu/mL) with PBS.

The respective agar assay plates were flooded with 1 mL of microbial suspension, excess inoculum was removed and the plates were allowed to completely dry before wells (6.5 mm diameter) were cut in the agar into which measured volumes (50-100 μL) of sample extract was added. 5 μL of a 0.00021 M tetracycline control was included. The plates were then incubated for 3 days and the inhibition zones were measured in mm. Samples were assayed in triplicate. Appropriate negative controls were included in all cases.

### Viable Counts Assay:

The activity of the sample extracts was assessed quantitatively by viable counts according to the following method. Microbial suspensions were prepared as described above, mixed in a 1:1 ratio with sample extract and then incubated under their respective growth conditions for 90 minutes. For the positive control the microbial suspensions were mixed with PBS. The samples were then processed for viable counts using standard procedures. Samples were assayed in duplicate/triplicate (3 times). Appropriate negative controls were included in all cases.

The results showed that while killing H. pylori the extract exhibited some toxicity towards the Gram-negative bacteria E. coli and S. typhiiuurium but had no notable effect on the Gram positive micro-organisms including the probiotic Lactobacillus acidophilus (FIG. 2). Being highly selective towards H. pylori indicates that the active components may offer potential as efficacious anti-H. pylori agents; killing H. pylori while having no significant adverse effect on the other gut micro flora.

### EXAMPLE 2

### The Effect of pH on the Bactericidal Activity of Potato Juice was Investigated

H. pylori is unique in the sense that it is the only known micro-organism capable of efficiently colonising the acidic gastric environment in the stomach (19). To do this the bacterium must first cross the acidic lumen which exhibits a pH value in the region of 1.4 before colonising the acidic gastric mucus where a more moderate pH range may fluctuate (20). Thus to further define the efficacy of potato juice as an effective anti-H. pylori agent it is important to establish whether the extract can maintain its bactericidal activity when exposed to lower pH values.

10 mL aliquots of potato juice were adjusted to a pH of 1.0, 3.0 and 5.0 with concentrated HCl and incubated for 4 hours with continuous shaking at 37° C. The solutions were then neutralised with 5 M NaOH, filtered through a 0.22 μm membrane and assayed for antibacterial activity against H. pylori by the viable count method. Negative controls consisted of 10 mL aliquots of water treated in the same way as the analytical samples. The experiment was done in dupliate, each of which contained 3 replicates.

The results showed that the extract lost its relative toxicity at pH 1.0 and 3.0 whereas at pH 5.0 the extract was still very active when compared to the negative control (see FIG. 3).

### EXAMPLE 3

### Effect of a Simulated Digest on the Bactericidal Activity of Potato Juice

Although it has been ascertained that the activity can survive at pH 5.0 (see Example 2) it was decided to investigate whether the activity can be maintained after exposure to a system which would mimic the stomach environment more closely. To do this a published procedure (21) with some modifications was used. 10 mL of potato juice was supplemented with 37 mM NaCl, 0.03 M HCl and 32 mg pepsin and incubated for 2 hours with continuous shaking at 37° C. The solution was then neutralised with 5 M NaOH, filtered through a 0.22 μm membrane and assayed for activity against H. pylori by the viable count method. A 10 mL water aliquot treated in the same way as the analytical sample served as a negative control. Each sample was assayed in triplicate.

The results showed that when potato juice was supplemented with 37 mM NaCl, 0.03 M HCl and 32 mg pepsin and incubated for 2 hours with continuous shaking at 37° C. activity was maintained when compared to the corresponding negative controls (FIG. 4). Pepsin is a digestive enzyme that is activated by HCl in the stomach and it preferentially cleaves at carboxylic groups of aromatic amino acids such as phenylalanine and tryptophan. It should be noted that in the case of potato juice the exposure was more drastic than that in the published study as in the latter case a 5% w/v extract of the herb Larrea divaricata was added to 100 mL of a simulated gastric fluid containing the above supplements.

### EXAMPLE 4

### EtOH Precipitation of the Crude Potato Juice Extract and Anion and Cation HPLC Fractionations of the EtOH Soluble Faction were Carried out to Purify the Antibacterial Activity of the Fraction

### EtOH Precipitation of Crude Product:

The crude potato juice extract was dark and rather dirty due to the presence of higher molecular weight polymers formed by the well established activity of polyphenol oxidase (PPO) in potato tissue (22). This led to an increased back pressure on the HLPC column so it was decided to do an EtOH precipitation of the crude extract with the aim of (a) further defining the activity and (b) obtaining a cleaner fraction for isolation of the antibacterial components. The procedure described by Bui et al. (23) was followed. EtOH precipitation is a common procedure used during the isolation of higher molecular weight components such as proteins and polysaccharides from biological matrices, whereby in most cases they precipitate out of solution upon addition of several volumes of alcohol to aqueous extracts.

Crude potato juice was prepared from 10.2 Kg of material as previously described to give 2,441 mL of extract. This extract was lyophilised and resuspended in water to give 360 mL of concentrated crude potato juice. For the precipitation 4 volumes of 96% EtOH was added to 1 volume of juice and left for 30 minutes. The solution was then centrifuged at 2,500 rpm for 20 minutes which gave an EtOH-water soluble fraction and an EtOH insoluble precipitate. The procedure was repeated 4 times and the combined EtOH soluble fraction (5.2 L) was concentrated to dryness in vacuo at ±40° C., resuspended in water, lyophlised and then resuspended in water to give 190 mL of concentrated extract. The EtOH insoluble precipitate was repeatedly washed with water and the combined supernatant (1.18 L) was lyophilised and resuspended in 50 mL water. All samples were stored at −80° C. until required for further analysis.

The results obtained showed that when compared to the crude potato juice the bulk of the activity was localised in the ethanol soluble fraction (see FIG. 5). This fraction was yellow and constituted a free flowing liquid. Furthermore it could be filtered more easily than the crude juice. The EtOH precipate itself was dark and clumpy like the concentrated crude potato juice indicating that most of the higher molecular weight dark polymers were present in this fraction.

This result is interesting because polysaccharides tend to be insoluble in EtOH whereas certain proteins such as promalines are soluble in 70% EtOH. Incidentally the final EtOH concentration in this case was 76% v/v.

### Ion Exchange Separation of the EtOH Soluble Fraction:

Proteins and other biomolecules possess a property known as a PI value which is the pH at which the molecule carries no net charge. At a pH above the PI value the molecule is negatively charged whereas at a pH below the PI value the molecule is positively charged. This property can be exploited during ion exchange chromatography. Basically ion exchange involves eluting an extract on a positively charged column (anion exchange) or on a negatively charged column (cation exchange), initially with a buffer such as Tris or MES to elute unbound material and then introducing a linear salt gradient to displace molecules bound to the column at the respective pH. In order to bind the molecules must possess a net charge opposite to that of the column at the pH used.

All separations were done on an ETTAN LC (Amersham Biosciences) equipped with a UV detector (215, 254 and 280 nm) and a conductivity recorder. The results were anlaysed using Unicorn™ software.

For the anion exchange fractionation, the column used was MonoQ HR 5/5 (1 mL volume), charged group: O—CH₂—CHOH—CH₂—O—CH₂—CHOH—CH₂—N⁺ (CH₃)₃. Mobile phase: Buffer A: 20 mM Tris-HCl (pH 8), Buffer B: 20 mM Tris in 2 M NaCl (pH 8). The column was eluted with 3.5 column volumes of 100% Buffer A to remove unbound material before introduction of a linear gradient mode going from 0% B to 100% B over 10 column volumes to displace bound material. The flow rate was between 0.25 to 0.4 mL.min⁻¹. Between 10 and 20×250 μL aliquots of each fraction (in buffer A) were delivered via a 500 μL injection loop. 30, 0.5 mL fractions were collected for each run and the fraction replicates were pooled, lyophilised and resuspended in 500 μL before assaying for anti-bacterial activity. Prior to assaying fractions 8 to 30 were desalted via the centrifugal filters (MWCO 3 kDa) according to the manufacturers instruction (VIVASCIENCE).

20, 250 μL aliquots of each fraction (in buffer A) were separated on the MonoQ column and fractions 2 to 7 (anion exchange flow through) were combined to give 60 mL of isolated anion exchange flow through for each of the EtOH soluble fractions. The bulk isolated anion exchange flow through was lyophilised and resuspended in 3 mL buffer A. The EtOH soluble fraction anion exchange flow through (3 mL) were separated by cation exchange as follows:

Column: MonoS HR 5/5 (1 mL), charged group: —O—CH₂—CHOH—CH₂—O—CH₂—CHOH—CH₂—SO₃. Mobile phase: the same as described for the MonoQ column. Flow rate: 0.5 mL.min⁻¹. Samples were delivered via a 1 mL injection loop. 30, 0.5 mL fractions were collected for each run and fractions 1 to 30 and 8 to 30 from the EtOH soluble fraction were desalted. The desalted fractions were processed as previously described.

Anion exchange separation of the EtOH soluble fraction found that the bulk of the activity was present in the flow through and thus did not stick to the column (see FIG. 6). Furthermore some degree of separation has occurred as the fractions containing the negatively charged material, fractions 10 to 17 (FIG. 7), were well separated from fractions 1 to 3, 6 and 7 (FIG. 7) which exhibited activity against H. pylori. This result shows that at pH 8.0 the antibacterial components are neutral or positively charged. However after bulk isolation of fractions 1 to 7 (anion exchange flow-through) and re-separation via cation exchange the active components appeared to have bound to the column as activity was present in fractions 11 to 14 which eluted after introduction of the salt gradient (FIG. 9).

The NaCl concentration at this point was between 307 to 721 mM. On the corresponding HPLC chromatogram (FIG. 8) there was a small peak present in fraction 12 that absorbed at 215, 254 and 280 nm indicating the possible presence of proteinaceous material. On the whole these results show that at pH 8.0 the antibacterial activity carries a net positive charge. In addition these results also show that the components appear to have been substantially purified as they are not present in the flow through where practically all of the UV absorbing material eluded (FIG. 8).

### EXAMPLE 5

### Phytochemical Analysis of the Cation Exchange Fractionated Antibacterial EtOH Soluble Fraction Anion Exchange Flow Through

It was previously determined in Example 4 that the antibacterial activity did not stick to the MonoQ anion exchange column at pH 8 and thus was present in the flow through. This flow through was then fractionated on a MonoS cation exchange column at pH 8 whereby the activity did bind to the column. The bulk of the activity was present in fraction 11 and there was tailing off of activity in fractions 12 to 15.

Fractions 1 to 15 were assayed for reducing sugars and protein via the phenol. sulphuric acid and BCA assays respectively and for primary amine groups by TLC spot tests with ninhydrin. The results are summarised in Table 1 below and illustrated in FIG. 10-12 of the accompanying drawings.

TABLE 1
Phytochemical analysis of the cation exchange fractionated
antibacterial EtOH soluble fraction anion exchange flow through.

| | Activity (% | Reducing | Protein | Primary amine |
|---|---|---|---|---|
| Fraction | cell growth) | sugars (mg) | (mg) | groups (—NH₂) |
| 1 | 100 | — | — | − |
| 2 | 100 | 3.15 | 0.022 | + |
| 3 | 100 | 10 | 0.063 | + |
| 4 | 100 | 0.47 | 0.052 | + |
| 5 | 56 | — | 0.029 | − |
| 6 | 17 | — | 0.02 | − |
| 7 | 92 | 0.007 | 0.02 | − |
| 8 | 90 | 0.009 | 0.025 | − |
| 10 | 100 | — | 0.027 | − |
| 11 | 0.5 | 0.017 | 0.031 | + |
| 14 | 15 | — | 0.024 | − |
| 15 | 21 | 0.003 | 0.004 | − |

−: not detected,
+: positive reaction

The results show that the antibacterial fraction, fraction 11 tested positive for reducing sugars, protein and contains components with primary amine groups (—NH₂). Primary amine groups are essential functional groups found in amino acids and hence, protein as well as in aminosugars.

Whilst the consumption of raw potato juice may alleviate the symptoms of gastritis it would clearly be impossible to maximize health benefits in this way due to the frequency and quantity of potato juice that would need to be consumed. The identification and isolation of a EtOH soluble fraction enables a concentrated amount of the bioactive part of the juice to be introduced into a suitable product (“functional food”) to enable the active part of the juice to be consumed in the necessary quantities.

### EXAMPLE 6

### Preparation of a Powder of a EtOH Soluble Fraction of Potato Juice

A EtOH soluble fraction of homogenized potato juice was prepared as described above and freeze-dried to form a powdered extract according to the invention.

### EXAMPLE 7

### Preparation of a Powder Mix of a EtOH Soluble Solution

3.0 g of freeze dried powdered extract (Example 2) was mixed with 0.5 g powdered citric acid, 26.3 g of granulated sugar and 0.2 g of a standard spray-dried mix of flavouring.

This mixture represents a free-flowing powder formulation (containing 3.0 g of the active fraction) that is suitable for packaging in a sachet. The powder mix may be diluted to taste and drunk when required by a subject suffering from gastritis

### EXAMPLE 8

### Preparation of an Orange Drink for Use According to the Invention

(a) 100 ml of crude preparation was mixed with 100 mls of double concentrate orange juice (orange juice concentrate diluted in water to double strength).
(b) 3.5 g of freeze-dried powder was dissolved in 100 mls of orange juice (or alternatively with orange juice concentrate and water).

The orange drink preparations (a or b) may be consumed by a subject immediately, refrigerated for later consumption or sealed in a bottle or carton for a longer shelf life.

It will be appreciated that orange juice may be readily substituted with a palatable alternative, such as a milk drink.

### EXAMPLE 9

### HPLC Finger Print Analysis of a Less than or Equal to 1 kDa to Greater Than or Equal to 500 Da Fraction

An anti-adhesive fraction, that is a fraction with less than or equal to 1 kDa to greater than or equal to 500 Da fraction, was analysed by reversed phase HPLC to provide a separation that would be directly suitable for LC-MS analysis without the presence of salt as is the case with the HPAEC HPLC chromatogram. The HPLC column used was a Gemini C₁₈ 250×4.6 mm i.d column with a particle size of 5 μm utilising a programme designed to separate the free phenolic compounds in potatoes (24).

The mobile phase consisted of A=water:methanol: acetic acid (88:10:2) and B=water:methanol: acetic acid (8:90:2) and involved an increasing gradient of B to 10% over 9 minutes followed by a sharp increase to 100% B between 9 to 13 minutes before returning to the initial conditions.

The results below show a typical trace obtained for the less than or equal to 1 kDa to greater than or equal to 500 Da fraction (ie ≦1 kDA to ≧500 Da) (FIG. 13a). When monitored at 280 nm there were several absorbing peaks but notably two, 1 and 2 appeared to have separated (FIG. 13a). The UV spectrum of peak 1 and 2 are shown in FIGS. 14a and b respectively. Both these components are aromatic in nature and could be purines, pyrimidines or aromatic amino acid residues. At 350 nm the HPLC chromatogram was different (FIG. 13b) with components eluting between 14 to 17 minutes. A close up of this region (FIG. 15) revealed several peaks. Although not completely resolved peaks A-C had UV spectra diagnostic of caffeic acid derivatives (FIG. 16a to c), phenolic phenylpropanoids belonging to the same family as chlorogenic acid (FIGS. 17 a and b), the predominant phenol in potatoes. No prominent peak was observed at 15 minutes which is when the chlorogenic acid marker eluted (FIG. 17a, b).

Therefore in summation there are at least 3 caffeic acid derivatives present in minor concentrations in the less than or equal to 1 kDa to greater than or equal to 500 Dalton fraction range KDa to z 500 Da) fraction, none of which are chlorogenic acid. Furthermore there are several aromatic components in the fraction of which two appear to have separated.

### REFERENCES

1. Blaser, M. J. (1998) Helicobacter pylori and gastric diseases. Biomed J. 316: 1507-1510.
2. Uemura, N., Okamoto, S., Yamamoto, S., Matsumura, N., Yamaguchi, S., Yamakido, M., Taniyama, K., and Sasaki, N., Schlemper, R. J. (2001) Helicobacter pylori infection and the development of gastric cancer. N. Engl. J. Med. 345: 784-789.
3. Guruge, J. L., Falk, P. G., Lorenz, R. G., Dans, M., Wirth, H. P., Blaser, M. J., Berg, D. E., and Gordon, J. I. (1998) Epithelial attachment alters the outcome of Helicobacter pylori infection. Proc, Natl, Acad, Sci. USA 95: 3925-3930
4. Censini, S., Stein, M., and Covacci, A. (2001) Cellular responses induced after contact with Helicobacter pylori. Curr. Opin. Microbiol. 4: 41-46
5. Calvert, X., Ducons, J., Bujanda, L., Bory, F., Montserrat, A., and Gisbert, J. P. (2005) Seven versus ten days of rabeprrazole triple therapy for Helicobacter pylori eradiction:amulticenter randomised trial. Am. J. Gastroenterol 100: 1696-1701
6. Goh, K. L. (2002) Update on the management of Helicobacter pylori infection, including drug resistant organisms. J Gastroenterol Hepatol 17: 482-487.
7. Kamiji, M. M., and de Oliveira R. B. (2005) Non-antibiotic therapies for Helicobacter pylori infection. Eur J Gastroenterol Hepatol 17: 973-981.
8. Galan, M. V., Kishan, A. A., Silverman, A. L., 2004. Oral broccoli sprouts for the treatment of Helicobacter pylori infection: a prelimary report. Digestive Diseases and Sciences 49, 1088-1090.
9. Fahey, J. W., Haristoy, X., Dolan, P. M., Kensler, T. W., Scholtus, I., Stephenson, K. K., Talalay, P., Lozneiwski, A., 2002. Sulforaphane inhibits extracellular, intracellular, and antibiotic-resistant strains of Helicobacter pylori and prevents benzo[a]pyrene induced stomach tumors. PNAS 99, 7610-7615.
10. Adeniyi, B. A., Anyiam, F. M., 2004. In vitro anti-Helicobacter pylori potential of methanol extract of Alluim ascalonicum Linn. (Liliacaea) leaf: susceptibility and effect on urease activity. Phytotherapy Research 18, 358-361.
11. Lin, Y. T., Kwon, Y. I., Labbe, R. G., Shetty, K., 2005. Inhibition of Helicobacter pylori and associated urease by oregano and cranberry phytochemical synergies. 71, 8558-8564.
12. Mahady, G. B., Pendland, S. L., Stoia, A., Chadwick, L. R., 2003. In vitro susceptibility of Helicobacter pylori to isoquinoline alkaloids from Sanguinaria Canadensis and Hydrastis canadensis. Phytotherapy Research 17, 217-221.
13. Wang, Y-C., Huang, T-L., 2005. Anti-Helicobacter pylori activity of Plumbago zeylanica L. FEMS Immunology and Medical Microbiology 43, 407-412.
14. Stamatis G., Kyriazopoulos, P., Golegou, S., Basayiannis, A., Skaltsas, S., Skaltsa, H., 2003. In vitro anti-Helicobacter pylori activity of Greek herbal medicines. Journal of Ethnopharmacology 88, 175-179.
15. Matsubara, S., Shibata, H., Ishikawa, F., Yokokura, T., Takahashi, M., Sugimura, T., Wakabayashi, K., 2003. Supression of Helicobacter pylori-induced gastritis by green tea extract in Mongolian gerbils. Bichemical and Biophysical Research Communications 310, 715-719.
16. O'Mahony, R., Al-Khtheeri, H., Weerasekera, D., Fernando, N., Vairi, D., Holton, J., Basset, C., 2005. Bactericidal and anti-adhesive properties of culinary and medicinal plants against Helicobacter pylori. World Journal of Gastroenterology 11, 7499-7507.
17. Deans, S. G., Ritchie, G., 1987. Antibacterial properties of plant essential oils. International Journal of Food Microbiology 5, 165-180.
18. Chou, L-S., Weimer, B., 1999. Isolation and characterization of acid- and bile-tolerant isolates from strains of Lactobacillus acidophilus. Journal of Dairy Science 82, 23-31.
19. Ha, N-C., Oh, S-T., Sung, J. Y., Cha, K. A., Lee, M. H., Oh, B-H., 2001. Supramolecular assembly and acid resistance of Helicobacter pylori urease. Nature Structural Biology 8, 505-509.
20. Stingl, K., De Reuse, H., 2005. Staying alive overdosed: how does Helicobacter pylori control urease activity? International Journal of Medical Microbiology 295, 307-315.
21. Stege, P. W., Davicino, R. C., Vega, A. E., Casali, Y. A., Correa, S., Micalizzi, B., 2006. Antimicrobial activity of aqueous extracts of Larrea divaricata Cav (jarilla) against Helicobacter pylori. Phytomedicine (In Press).
22. Friedman, M., Bautista, F. F., 1995 Inhibition of polyphenol oxidase by thiols in the absence and presence of potato tissue suspensions. Journal of Agricultural and Food Chemistry 43, 69-76.
23. Bui, A. K. T., Bacic, A., Pettolino, F., 2006. Polysaccharide composition of the fruit juice of Morinda citrifolia (Noni). Phytochemistry 67, 1271-1275.
24. Verde-Mendez, C, Delgado, M. A. R., Rodriguez, E. M. R., Romero, C. D., 2004. Content and free phenolic compounds in cultivars of potatoes harvested in Tenerife (Canary Islands). J. Agric. Food Chem. 52, 1323-1327.

1. A method of making a composition for use in the prevention or treatment of gastrointestinal disease, the method comprising the steps of adding an alcohol to potato juice to form an alcohol soluble fraction and an alcohol insoluble precipitate and recovering the alcohol soluble fraction.

2. A method according to claim 1 wherein the alcohol is ethanol.

3. A method according to claim 2 wherein at least 70% ethanol is used for the precipitation.

4. A method according to claim 3 wherein 96% ethanol is used for the precipitation.

5. A method according to any one of claims 1 to 4 wherein concentrated potato juice is subjected to at least two precipitations with the alcohol.

6. A method according to claim 5 wherein the potato juice is subjected to at least four precipitations with alcohol.

7. A method according to any one of claims 1 to 6 wherein the alcohol soluble fraction is further purified by means of ion exchange chromatography.

8. A method according to claim 7 wherein the alcohol soluble fraction is treated with anion HPLC at pH 8 to bind negatively charged components, collecting the non-bound components and treating said non-bound components with cation HPLC at pH 8 to bind positively charged components, eluting the positively bound components and collecting the eluted fraction.

9. A method according to any one of claims 1 to 8 wherein the composition comprises an amount of a less than or equal to 3 kDa soluble fraction derivable from potato juice.

10. A method according to any one of claims 1 to 9 wherein the composition comprises an amount of an equal to or greater than 500 Da to less than or equal to 1 kDa soluble fraction derivable from potato juice.

11. A method according to claims 10 wherein the greater than 500 Da to less than or equal to 1 kDa soluble fraction comprises three caffeic acid derivatives and two aromatic derivatives.

12. A composition prepared by a method according to any one of claims 1 to 11 for use in the prevention or treatment of gastrointestinal disease wherein the alcohol soluble fraction is provided in a therapeutically effective amount.

13. A composition according to claim 12 wherein the fraction includes at least part of a protein or polypeptide

14. A composition according to claim 12 or 13 wherein the fraction contains reducing sugars and/or components with primary amino groups.

15. A composition according to any one of claims 12 to 14 in powder form.

16. A composition according to claim 15 wherein the powder comprises a spray-dried or freeze-dried fraction.

17. A composition according to any one of claims 12 to 16 further comprising at least one of flavouring, sugars, sweetener, anti-oxidants, minerals or vitamins.

18. A composition according to any one of claims 12 to 17 for the prevention and/or treatment of gastric or duodenal ulcers and gastric or duodenal carcinomas.

19. Use of an alcohol soluble fraction derivable from potato juice for the manufacture of a medicament for the prevention or treatment of Gastrointestinal Disease.

20. Use of an alcohol soluble fraction derisible from potato juice according to claim 19 wherein the medicament is a composition according to any one of claims 12 to 18 in combination with a pharmaceutically acceptable carrier.

21. Use of an alcohol soluble fraction derisible from, potato juice according to claim 19 or 20 wherein the medicament is a liquid, capsule or tablet for oral consumption.

22. A method for the treatment of gastrointestinal disease comprising administering to a subject in need of such treatment a therapeutically effective amount of an alcohol soluble fraction derivable from potato juice.

23. A nutritional product for use in the prevention or treatment of gastrointestinal disease comprising a therapeutically effective amount of an alcohol soluble fraction derivable from potato juice.

24. A nutritional product according to claim 23 in the form of a beverage or drink.

25. A nutritional product according to claim 23 in the form of a diary product.

26. A nutritional product according to claim 23 comprising between 1 g/100 ml and 30 g/100 ml of the fraction.

27. A nutritional product according to claim 23 in the form of a powder or powder mix.

28. A nutritional product according to claim 23 in the form of a food bar or other solid food stuff.

29. An anti-bacterial agent comprising an alcohol soluble fraction derivable from potato juice.

30. An anti-bacterial agent as claimed in claim 29 wherein the fraction is positively charged.

31. An anti-bacterial agent according to claim 29 or 30 wherein the agent is active against H. pylori.

32. A method of making an anti-bacterial agent comprising the steps of:
preparing an aqueous extract of potato juice;
adding an alcohol to the aqueous extract to form an alcohol soluble fraction and an alcohol soluble precipitate;
recovering the alcohol soluble fraction;
treating said fraction with an anion exchange resin to bind negatively charged components and collecting the non-bound components from the resin;
treating said non-bound components with a cation exchange resin to associate positively-charged components with said resin;
eluting said positively-charged components from said resin with an eluting solution to obtain an eluate having anti-bacterial properties;
collecting and, optionally concentrating, said eluate.
